(19) United States
(12) Patent Application Publication
(10) Pub. No.: US 2011/0137105 A1
(43) Pub. Date: Jun. 9, 2011

(54) Portable magnetic field stimulator

(76) Inventors: Chih-Kuo Liang, Yung-Kang City (TW); Shih-Jui Yang, Taichung City (TW)

(21) Appl. No.: 12/591,962

(22) Filed: Dec. 7, 2009

(51) Int. Cl. A61N 2/04; H03K 3/80

(52) U.S. Cl. 600/13; 307/106

(57) ABSTRACT

The portable magnetic field stimulator of the present invention has a microcontroller which sets sinusoidal wave or pulse voltage signals of different frequency, intensity, duty cycle through a manipulation interface, and inputs the signals to a pulse type voltage/current conversion circuit or sinusoidal wave type voltage/current conversion circuit. Finally, the current is transported to the stimulating coil to produce sinusoidal wave or pulse wave magnetic field having direct current level with frequency 1˜2 kHz and with magnetic flux 1˜300 Gauss. Thereby, not only the variation of frequency can be controlled but also the other subtle parameters such as duty cycle, multi wave form switching are also able to be manipulated and set, and different stimulating wave form of sinusoidal wave or pulse can also be produced. In addition, it can be applied in diversified symptoms.

### BACKGROUND OF THE INVENTION

1. Field of the Invention

The present invention relates to a portable magnetic field stimulator, particularly to a novel portable magnetic field stimulator which can produce sinusoidal wave or pulse wave voltage signals of different frequency, strength, duty cycle, and can produce sinusoidal wave or pulse wave magnetic field having direct current level with a frequency of 1˜2 kHz and with a magnetic flux of 1˜300 Gauss, such that it can be widely applied in diversified symptoms. It can increase more practical effectiveness in the integral embodiment and usage thereof.

2. Brief Description of Prior Art

According to the law of physics, change of magnetic field will produce induced electric field and induced current. Inasmuch as magnetic field is not baffled by body organization, electric current can be produced in the interior of body by this principle. Comparing with functional electric stimulation, functional magnetic stimulation can excite body organization more deeply without causing any uncomfortableness, and can be rendered to human body in non-contact mode. Magnetic stimulation is often used in the stimulation of spinal nerve. According to many research, when magnetic stimulator is placed over high position such as spinal, induced electric current will be present at nerve foramina of upper and lower three segments of spinal, i.e., the positions of spinal nerve. Several physiological functions such as respiration, intestine and stomach activity are regulated and controlled by several segments of spinal nerve. Therefore, only if the coil of the magnetic stimulator is placed over certain one segment of spinal, induced electric current will be produced on several spinal nerves. In fact, induced electric current is present not only on the nerve, but also on the organization within the range of induced electric field. Consequently, muscles within the electric field also produce apparent induced electric current. Sufficiently big electric current intensity will cause shrinking of muscles. Furthermore, as already manifested in many research conducted in home and abroad as well, when the frequency of the electromagnetic field is set at low frequency and the magnetic field intensity is set in the range of window bioeffect capable of triggering bioresponse, non-thermal effect will be produced with respect to cell organization to cause rising of the concentration of calcium ion within cell. This will facilitate the heal over of bone.

As to the magnetic stimulators available on market, pulse type is mostly used in their structures to conduct magnetic stimulation for users. It is found in their total implementation and usage that there is still some space for improvement due to the disadvantages list below:

1. In the above structure, there are few variable items on stimulating parameter. Only the variation of frequency can be controlled. The other subtle parameters such as duty cycle, multi wave form switching is not included in the controllable parameter items.

2. Their application field is unitary, most of them are only developed for the facilitation of bone heal over, and are unable to be applied on different symptoms.

3. Most of the internal circuit in the magnetic field stimulator utilizes RC charge and discharge method for producing magnetic field such that only sole stimulating wave form can be produced.

In view of the above facts, the inventor of the present invention innovates a portable magnetic field stimulator according to the improvement conducted on defects of the conventional structure based on his abundant experience of R&D and manufacturing in relevant field so as to attain the goal of obtaining better practical effectiveness and features.

### SUMMARY OF THE INVENTION

The portable magnetic field stimulator of the present invention essentially has a microcontroller served as core part which sets sinusoidal wave or pulse voltage signals of different frequency, intensity, duty cycle through a manipulation interface, and inputs the signals to a pulse type voltage/current conversion circuit or a sinusoidal wave type voltage/current conversion circuit. Finally, the current is transmitted to a stimulating coil to produce sinusoidal wave or pulse wave magnetic field having direct current level with a frequency of 1˜2 kHz and with a magnetic flux of 1˜300 Gauss. Thereby, not only the variation of frequency can be controlled but also the other subtle parameters such as duty cycle, multi wave form switching are also able to be manipulated and set, and different stimulating wave form of sinusoidal wave or pulse can also be produced. In addition, it can be applied in diversified symptoms. It can increase more practical effectiveness in the integral implementation and usage thereof.

### BRIEF DESCRIPTION OF THE ACCOMPANYING DRAWINGS

FIG. 1 is a schematic block diagram showing the portable magnetic field stimulator of the present invention.

FIG. 2 is an output wave form of the portable magnetic field stimulator of the present invention.

FIG. 3 is another output wave form of the portable magnetic field stimulator of the present invention.

### DETAILED DESCRIPTION OF PREFERRED EMBODIMENT

The technical content, objects and effectiveness of the present invention will become more apparent by the detailed description of a preferred embodiment of the present invention in conjunction with the accompanying drawings.

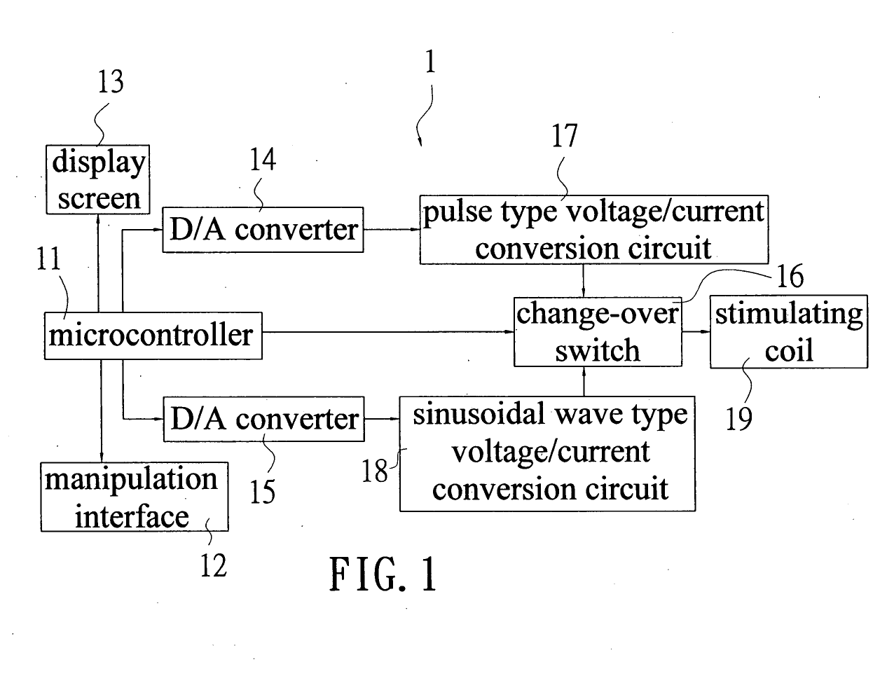

FIG. 1 is a schematic block diagram showing the portable magnetic field stimulator of the present invention. As shown in FIG. 1, the magnetic field stimulator (1) is essentially provided with a microcontroller (11). A manipulation interface (12) is connected to the microcontroller (11) for users to conduct the setting and manipulating for each item. A display screen (13) is connected with the microcontroller (11) so as to display the status of each manipulation. D/A converters (14), (15) and a change-over switch (16) are also respectively connected to the microcontroller (11), in which the D/A converter (14) is connected with a pulse type voltage/current conversion circuit (17), while the D/A converter (15) with a sinusoidal wave type voltage/current conversion circuit (18). The pulse type voltage/current conversion circuit (17) and the sinusoidal wave type voltage/current conversion circuit (18) are both connected to the change-over switch (16) which is in turn connected to a stimulating coil (19).

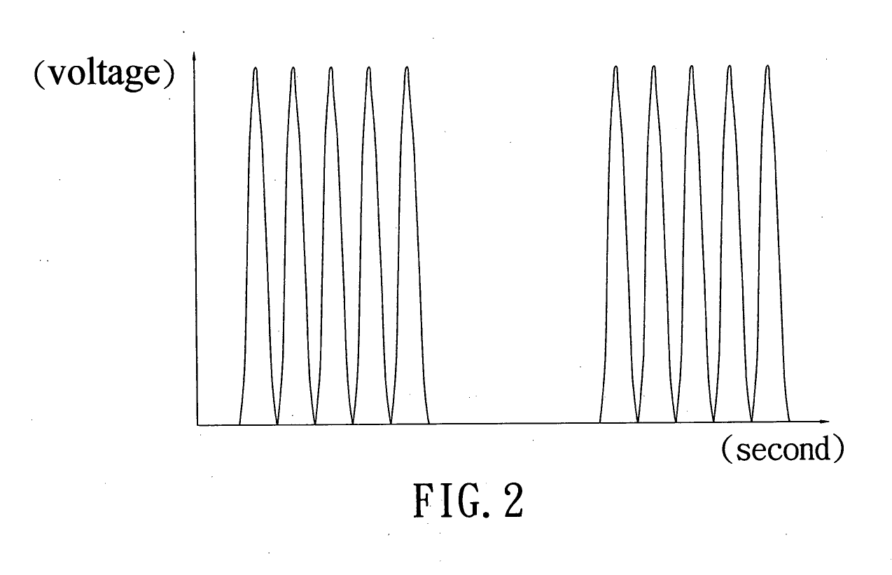

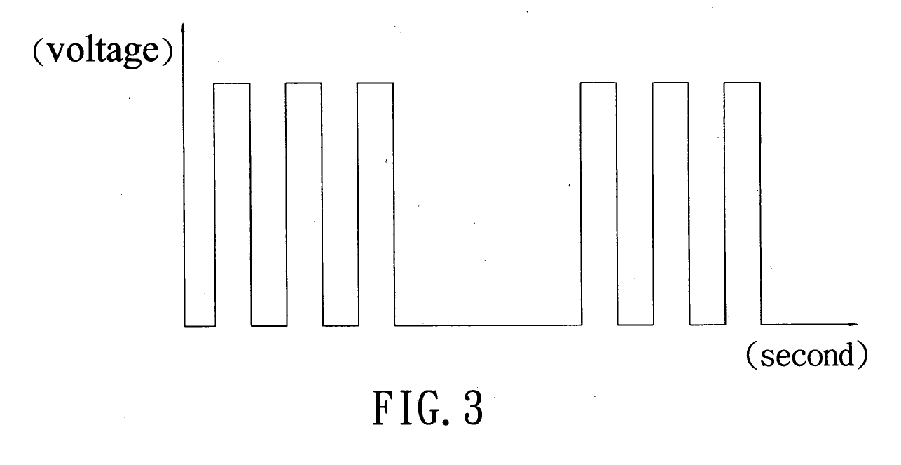

During use of the magnetic field stimulator (1), users conduct the setting and manipulating of each item for the microcontroller (11) by the manipulation interface (12), and the status of setting and manipulating is displayed on the display screen (13) through the microcontroller (11). The microcontroller (11) controls the D/A converters (14),(15), according to the status of setting and manipulating conducted by users, through serial transmission so as to produce sinusoidal wave or pulse wave voltage signals of different frequency, intensity and duty cycle. These signals are then inputted to the pulse type voltage/current conversion circuit (17) or the sinusoidal wave type voltage/current conversion circuit (18), and the current signals of the pulse type voltage/current conversion circuit (17) or the sinusoidal wave type voltage/current conversion circuit (18) is transmitted to the stimulating coil (19) so as to produce sinusoidal wave (referring to the output wave form diagram shown in FIG. 2) or pulse wave (referring to the output wave form diagram shown in FIG. 3) magnetic field having direct current level with a frequency of 1˜2 kHz and with a magnetic flux of 1˜300 Gauss.

The above embodiment or drawings are not intended to restrict the structural aspect or dimension of the present invention. Variations of modifications made by the person having ordinary skill in the art without departing from the spirit and scope of the present invention are still regarded to be within the scope of the present invention.

It can be understood based on the foregoing, the portable magnetic field stimulator of the present invention essentially has a microcontroller as the core part which sets sinusoidal wave or pulse voltage signals of different frequency, intensity, duty cycle through a manipulation interface, and inputs the signals to pulse type voltage/current conversion circuit or sinusoidal wave type voltage/current conversion circuit. Finally, the current is transported to the stimulating coil to produce sinusoidal wave or pulse wave magnetic field having direct current level with a frequency of 1˜2 kHz and with a magnetic flux of 1˜300 Gauss. Thereby, not only the variation of frequency can be controlled but also the other subtle parameters such as duty cycle, multi wave form switching are also able to be manipulated, controlled and set, and different stimulating wave form of sinusoidal wave or pulse can also be produced. In addition, it can be applied in diversified symptoms widely. It can increase more practical effectiveness in the integral implementation and usage thereof.

What is claimed is:

1. A portable magnetic field stimulator, wherein it is essentially provided with a microcontroller, a manipulation interface being connected to said microcontroller for users to conduct setting and manipulating on each item, a display screen being connected to said microcontroller so as to display the status of setting and manipulating on each item, D/A converters and a change-over switch being also connected to said microcontroller, one of the D/A converters being connected to a pulse type voltage/current conversion circuit, while the other of the D/A converters being connected to a sinusoidal type voltage/current conversion circuit, said pulse type voltage/current conversion circuit and said sinusoidal type voltage/current conversion circuit being connected to said change-over switch which is then connected to a stimulating coil.

2. The portable magnetic field stimulator as claimed in claim 1, wherein said stimulating coil produces a wave form with a frequency of 1˜2 kHz.

3. The portable magnetic field stimulator as claimed in claim 1, wherein said stimulating coil produces a wave form with a magnetic flux of 1˜300 Gauss.
